(19) United States
(12) Patent Application Publication
(10) Pub. No.: US 2011/0091882 A1
(43) Pub. Date: Apr. 21, 2011

(54) DETERMINATION OF METHYLATION STATUS OF POLYNUCLEOTIDES

(75) Inventors: Edward N. Granados, Vernon, IL (US); Thomas G. Laffler, Libertyville, IL (US)

(73) Assignee: IBIS BIOSCIENCES, INC., Carlsbad, CA (US)

(21) Appl. No.: 12/896,574

(22) Filed: Oct. 1, 2010

Related U.S. Application Data
(60) Provisional application No. 61/248,206, filed on Oct. 2, 2009.

(51) Int. Cl. C12Q 1/68; C12M 1/34

(52) U.S. Cl. 435/6; 435/287.2

(57) ABSTRACT

The present invention provides compositions and methods for detecting the methylation status of a nucleic acid. In particular, the present invention provides a mass spectrometry-based method of determining DNA methylation status without sequencing.

The present application claims priority to U.S. Provisional Patent Application Ser. No. 61/248,206, filed Oct. 2, 2009, the disclosure of which is herein incorporated by reference in its entirety.

### FIELD OF THE INVENTION

The present invention provides compositions and methods for detecting the methylation status of a nucleic acid. In particular, the present invention provides a mass spectrometry-based method of determining DNA methylation status without sequencing.

### BACKGROUND

DNA methylation is a type of chemical modification of DNA that can be inherited and subsequently removed without changing the DNA sequence. As such, it is part of the epigenetic code (Jaenisch & Bird. (2003) Nature Genetics, 33, 245, herein incorporated by reference in its entirety). DNA methylation involves the addition of a methyl group to a DNA nucleobase. In the most common example, a methyl group is added to the number 5 carbon of the cytosine pyrimidine ring. Cytosine methylation generally has the effect of reducing gene expression. Methylation is a common capability of all viruses for self non-self identification. DNA methylation at the 5 position of cytosine has been found in every vertebrate examined. In adult somatic tissues, DNA methylation typically occurs in a CpG dinucleotide context; non-CpG methylation is prevalent in embryonic stem cells (Dodge et al. (2002) Gene 289 (1-2): 41-48, Haines et al. (2001) Developmental Biology 240 (2): 585-598, herein incorporated by reference in their entireties). In plants, cytosines are methylated both symmetrically (CpG or CpNpG) and asymmetrically (CpNpNp). Long term memory storage in humans may be regulated by DNA methylation (Miller & Sweatt. (2007 Mar. 15) Neuron 53 (6): 857-869, Powell & Devin. (2008) New Scientist, herein incorporated by reference in their entireties).

In mammals, DNA methylation is essential for normal development and is associated with a number of key processes including imprinting, X-chromosome inactivation, suppression of repetitive elements and carcinogenesis. Between 60-90% of all CpGs are methylated in mammals (Tucker. (2001) Neuron. 30(3): 649-52, herein incorporated by reference in its entirety). CpGs are grouped in clusters called “CpG islands” that are present in the 5′ regulatory regions of many genes. In many disease processes such as cancer, gene promoter CpG islands acquire abnormal hypermethylation, which results in heritable transcriptional silencing.

Methylation analysis has been accomplished by the field using several different methods. Bisulfite conversion or Methylation Sensitive Restriction Enzyme (MSRE) is an element of all the technologies. Bisulfite conversion methods rely on sequencing, primer-probes, primer-gel, or primer-array analysis. A disadvantage to all these methods is the complexity of the methods and/or the lack of detailed information regarding the exact numbers of methylated residues in regions of interest.

A method for analyzing DNA for 5-methylcytosine is based on the specific reaction of bisulfite with cytosine which, upon subsequent alkaline hydrolysis, is converted to uracil which corresponds to thymidine in its base pairing behavior. 5-methylcytosine remains unmodified under these conditions. Consequently, the original DNA is converted in such a manner that methylcytosine, which originally cannot be distinguished from cytosine in its hybridization behavior, can now be detected, for example, by amplification and hybridization or sequencing. These techniques are based on base pairing which is now taken full advantage of.

An overview of the further known possibilities of detecting 5-methylcytosines can be gathered from the following survey article: Rein, T., DePamphilis, M. L., Zorbas, H., Nucleic Acids Res. 1998, 26, 2255.

The bisulfite technology has involved short specific fragments of a known gene which are amplified subsequent to a bisulfite treatment and either completely sequenced (Olek, A. and Walter, J., Nat. Genet. 1997, 17, 275-276) or individual cytosine positions are detected by a primer extension reaction (Gonzalgo, M. L., and Jones, P. A., Nucl. Acids Res. 1997, 25, 2529-2531, WO 9500669) or by an enzymatic digestion (Xiong, Z. and Laird, P. W., Nucl. Acids. Res. 1997, 25, 2532-2534). In addition, the detection by hybridization has also been described (Olek et al., WO 99 28498).

Further publications dealing with the use of the bisulfite technique for methylation detection in individual genes are: Xiong, Z. and Laird, P. W. (1997), Nucl. Acids Res. 25, 2532; Gonzalgo, M. L. and Jones, P. A. (1997), Nucl. Acids Res. 25, 2529; Grigg, S. and Clark, S. (1994), Bioassays 16, 431; Zeschnik, M. et al. (1997), Human Molecular Genetics 6, 387; Teil, R. et al. (1994), Nucl. Acids Res. 22, 695; Martin, V. et al. (1995), Gene 157, 261; WO 97 46705; WO 95 15373 and WO 45560, herein incorporated by reference in their entireties. Using the bisulfate technique for detecting cytosine methylation in DNA samples is described in U.S. Pat. No. 7,524,629, herein incorporated by reference in its entirety.

MSRE PCR methods suffer from the fact that if more than one MSRE site is present in the region of interest, for example multiple Acil sites, then all of the Acil sites must be methylated for detection to occur. Cleavage of a single unmethylated site will result in a negative result. Moreover, in order to accurately determine the total methylation status, more than one MSRE with different specificities may be necessary. As the number of MSRE's are increased, so increases the probability of false negatives. The MSRE approach also suffers from difficulties caused by incomplete digestions, which can result in false positives. In addition to the above limitations, MSRE are costly, may deteriorate over time, and are highly dependent on concentration and digestion conditions. Some MSRE methods also lack specificity with respect to cutting.

Bisulfite PCR methods utilize gels, probes, or arrays for analysis. Bisulfite PCR methods which utilize gels do not provide information regarding methylation content. Bisulfite PCR methods which utilize probes can suffer from being insensitive to mismatches. Inaccurate determination may occur as a result of mismatches. PCR probe assays are somewhat restricted in terms of the maximum usable amplicon size. Multiplexing becomes difficult in multiprobe assays to due increased probability of primer probes interactions.

What is needed are new methods and systems for detecting and characterizing methylation status of nucleic acid molecules.

### SUMMARY

In some embodiments, the present invention provides a method of determining the methylation status of a nucleic acid, the method comprising: reacting a nucleic acid molecule with bisulfate, amplifying one or more segments the nucleic acid using at least one purified oligonucleotide primer pair to produce an amplification product, and determining the mass or base composition of the amplification product, thereby determining said methylation status of said nucleic acid. In some embodiments, the nucleic acid comprises DNA. In some embodiments, the nucleic acid is GC-rich. In some embodiments, amplifying comprises PCR. In some embodiments, detecting the amplification product comprises detecting a molecular mass of the amplification product. In some embodiments, detecting the amplification product comprises determining a base composition of the amplification product, wherein the base composition identifies the number of A residues, C residues, T residues, G residues, U residues, analogs thereof and/or mass tag residues thereof in the amplification product, whereby the base composition indicates the methylation status of the nucleic acid. In some embodiments, the base composition indicates the methylation status of the nucleic acid through comparison of the base composition of the amplification product to calculated or measured base compositions of amplification products present in a database with the proviso that sequencing of the amplification product is not used to indicate the methylation status, wherein a match between the determined base composition and the calculated or measured base composition in the database indicates methylation status. In some embodiments, the base composition indicates the methylation status of the nucleic acid through comparison of the base composition of the amplification product to the base composition of a control nucleic acid with the proviso that sequencing of the amplification product is not used to indicate the methylation status, wherein differences in mass between the determined base composition and control base composition indicates methylation status. In some embodiments, the present invention comprises an initial step of isolating nucleic acid from a subject or sample.

Various amplification, mass and/or base composition determination, data analysis, and nucleic acid isolation and preparation methods, compositions, and systems may be employed. In some embodiments, the methods, compositions, and systems are those described in U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; 20080160512, 20080311558, 20090004643, 20090047665, 20090125245, 20090148829, 20090148836, 20090148837, 20090182511, WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222, Ecker et al. (2005) “The Microbial Rosetta Stone Database: A compilation of global and emerging infectious microorganisms and bioterrorist threat agents” BMC Microbiology 5(1):19; Ecker et al. (2006) “The Ibis T5000 Universal Biosensor: An Automated Platform for Pathogen Identification and Strain Typing” JALA 6 (10:341-351; Ecker et al. (2006) “Identification of Acinetobacter species and genotyping of Acinetobacter baumannii by multilocus PCR and mass spectrometry” J Clin Microbiol. 44(8):2921-32; Ecker et al. (2005) “Rapid identification and strain-typing of respiratory pathogens for epidemic surveillance” Proc Natl Acad Sci USA. 102(22):8012-7; Hannis et al. (2008) “High-resolution genotyping of Campylobacter species by use of PCR and high-throughput mass spectrometry” J Clin Microbiol. 46(4):1220-5; Blyn et al. (2008) “Rapid detection and molecular serotyping of adenovirus by use of PCR followed by electrospray ionization mass spectrometry” J Clin Microbiol. 46(2):644-51; Sampath et al. (2007) “Global surveillance of emerging Influenza virus genotypes by mass spectrometry” PLoS ONE 2 (5):e489; Sampath et al. (2007) “Rapid identification of emerging infectious agents using PCR and electrospray ionization mass spectrometry” Ann N Y Acad. Sci. 1102:109-20; Hall et al. (2005) “Base composition analysis of human mitochondrial DNA using electrospray ionization mass spectrometry: a novel tool for the identification and differentiation of humans” Anal Biochem. 344(1):53-69; Hofstadler et al. (2003) “A highly efficient and automated method of purifying and desalting PCR products for analysis by electrospray ionization mass spectrometry” Anal Biochem. 316:50-57; Hofstadler et al. (2006) “Selective ion filtering by digital thresholding: A method to unwind complex ESI-mass spectra and eliminate signals from low molecular weight chemical noise” Anal Chem. 78(2):372-378; and Hofstadler et al. (2005) “TIGER: The Universal Biosensor” Int J Mass Spectrom. 242(1):23-41, each of which is herein incorporated by reference in its entirety.

### DESCRIPTION OF FIGURES

The foregoing summary and detailed description may be better understood when read in conjunction with the accompanying drawings which are included by way of example and not by way of limitation.

FIG. 1 shows a flow chart depicting an embodiment of the present invention performed on a methylated and unmethylated DNA sequence. The designated PCR primer regions are shown in gray while the probe regions are in black, nucleotides corresponding sequentially to C's from the original strands are underlined, methylated C's are designated as C^{m}.

### DEFINITIONS

It is to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this invention pertains. In describing and claiming the present invention, the following terminology and grammatical variants will be used in accordance with the definitions set forth below.

As used herein, the term “about” means encompassing plus or minus 10%. For example, “about 200 nucleotides” refers to a range encompassing between 180 and 220 nucleotides.

As used herein, the term “amplicon” refers to a nucleic acid generated using primer pairs. The amplicon is typically double stranded DNA; however, it may be RNA and/or DNA:RNA. The amplicon comprises DNA complementary to a sample nucleic acid. In some embodiments, primer pairs are configured to generate amplicons from a sample nucleic acid. As such, the base composition of any given amplicon may include the primer pair, the complement of the primer pair, and the region of a sample nucleic acid that was amplified to generate the amplicon. One skilled in the art understands that the incorporation of the designed primer pair sequences into an amplicon may replace the native sequences at the primer binding site, and complement thereof. In certain embodiments, after amplification of the target region using the primers the resultant amplicons having the primer sequences are used for subsequent analysis (e.g. base composition determination). In some embodiments, the amplicon further comprises a length that is compatible subsequent analysis.

Amplicons typically comprise from about 15 to about 200 consecutive nucleobases (i.e., from about 15 to about 200 linked nucleosides). One of ordinary skill in the art will appreciate that this range expressly embodies compounds of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, and 200 nucleobases in length. One of ordinary skill in the art will further appreciate that the above range is not an absolute limit to the length of an amplicon, but instead represents a preferred length range. Amplicon lengths falling outside of this range are also included herein so long as the amplicon is amenable to calculation of a base composition signature as herein described.

The term “amplifying” or “amplification” in the context of nucleic acids refers to the production of multiple copies of a polynucleotide, or a portion of the polynucleotide, typically starting from a small amount of the polynucleotide (e.g., as few as a single polynucleotide molecule), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR) are forms of amplification. Amplification is not limited to the strict duplication of the starting molecule. For example, the generation of multiple cDNA molecules from a limited amount of RNA in a sample using reverse transcription (RT)-PCR is a form of amplification. Furthermore, the generation of multiple RNA molecules from a single DNA molecule during the process of transcription is also a form of amplification.

As used herein, the term “base composition” refers to the number of each residue comprised in an amplicon or other nucleic acid, without consideration for the linear arrangement of these residues in the strand(s) of the amplicon. The amplicon residues comprise, adenosine (A), guanosine (G), cytidine, (C), (deoxy)thymidine (T), uracil (U), inosine (I), nitroindoles such as 5-nitroindole or 3-nitropyrrole, dP or dK (Hill F et al., Polymerase recognition of synthetic oligodeoxyribonucleotides incorporating degenerate pyrimidine and purine bases. Proc Natl Acad Sci USA. 1998 Apr. 14; 95(8):4258-63), an acyclic nucleoside analog containing 5-nitroindazole (Van Aerschot et al., Nucleosides and Nucleotides, 1995, 14, 1053-1056), the purine analog 1-(2-deoxy-beta-D-ribofuranosyl)-imidazole-4-carboxamide, 2,6-diaminopurine, 5-propynyluracil, 5-propynylcytosine, phenoxazines, including G-clamp, 5-propynyl deoxy-cytidine, deoxy-thymidine nucleotides, 5-propynylcytidine, 5-propynyluridine and mass tag modified versions thereof, including 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, 6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹⁵N and ¹³C. In some embodiments, the non-natural nucleosides used herein include 5-propynyluracil, 5-propynylcytosine and inosine. In some embodiments, the base composition for an unmodified DNA amplicon is notated as A_{w}GₓC_{y}T_{z}, wherein w, x, y and z are each independently a whole number representing the number of said nucleoside residues in an amplicon. Base compositions for amplicons comprising modified nucleosides are similarly notated to indicate the number of said natural and modified nucleosides in an amplicon.

As used herein, the term “base composition signature” refers to the base composition generated by any one particular amplicon.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

As used herein, the term “hybridization” or “hybridize” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, the melting temperature (Tₘ) of the formed hybrid, and the G:C ratio within the nucleic acids. A single molecule that contains pairing of complementary nucleic acids within its structure is said to be “self-hybridized.” An extensive guide to nucleic hybridization may be found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993), which is incorporated by reference.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method.

In some embodiments of the invention, oligonucleotide primer pairs can be purified. As used herein, “purified oligonucleotide primer pair,” “purified primer pair,” or “purified” means an oligonucleotide primer pair that is chemically-synthesized to have a specific sequence and a specific number of linked nucleosides. This term is meant to explicitly exclude nucleotides that are generated at random to yield a mixture of several compounds of the same length each with randomly generated sequence. As used herein, the term “purified” or “to purify” refers to the removal of one or more components (e.g., contaminants) from a sample.

As used herein, the term “molecular mass” refers to the mass of a compound as determined using mass spectrometry, for example, ESI-MS. Herein, the compound is preferably a nucleic acid. In some embodiments, the nucleic acid is a double stranded nucleic acid (e.g., a double stranded DNA nucleic acid). In some embodiments, the nucleic acid is an amplicon. When the nucleic acid is double stranded the molecular mass is determined for both strands. In one embodiment, the strands may be separated before introduction into the mass spectrometer, or the strands may be separated by the mass spectrometer (for example, electro-spray ionization will separate the hybridized strands). The molecular mass of each strand is measured by the mass spectrometer.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, the term “nucleobase” is synonymous with other terms in use in the art including “nucleotide,” “deoxynucleotide,” “nucleotide residue,” “deoxynucleotide residue,” “nucleotide triphosphate (NTP),” or deoxynucleotide triphosphate (dNTP). As is used herein, a nucleobase includes natural and modified residues, as described herein.

An “oligonucleotide” refers to a nucleic acid that includes at least two nucleic acid monomer units (e.g., nucleotides), typically more than three monomer units, and more typically greater than ten monomer units. The exact size of an oligonucleotide generally depends on various factors, including the ultimate function or use of the oligonucleotide. To further illustrate, oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Typically, the nucleoside monomers are linked by phosphodiester bonds or analogs thereof, including phosphorothioate, phosphorodithioate, phosphoroselenoate, phosphorodiselenoate, phosphoroanilothioate, phosphoranilidate, phosphoramidate, and the like, including associated counterions, e.g., H⁺, NH₄⁺, Na⁺, and the like, if such counterions are present. Further, oligonucleotides are typically single-stranded. Oligonucleotides are optionally prepared by any suitable method, including, but not limited to, isolation of an existing or natural sequence, DNA replication or amplification, reverse transcription, cloning and restriction digestion of appropriate sequences, or direct chemical synthesis by a method such as the phosphotriester method of Narang et al. (1979) Meth Enzymol. 68:90-99; the phosphodiester method of Brown et al. (1979) Meth Enzymol. 68:109-151; the diethylphosphoramidite method of Beaucage et al. (1981) Tetrahedron Lett. 22:1859-1862; the triester method of Matteucci et al. (1981) J Am Chem Soc 103:3185-3191; automated synthesis methods; or the solid support method of U.S. Pat. No. 4,458,066, entitled “PROCESS FOR PREPARING POLYNUCLEOTIDES,” issued Jul. 3, 1984 to Caruthers et al., or other methods known to those skilled in the art. All of these references are incorporated by reference.

As used herein a “sample” refers to anything capable of being analyzed by the methods provided herein. In some embodiments, the sample comprises or is suspected to comprise one or more nucleic acids capable of analysis by the methods. In certain embodiments, for example, the samples comprise nucleic acids (e.g., DNA, RNA, cDNAs, etc.). Samples can include, for example, blood, semen, saliva, urine, feces, rectal swabs, and the like. In some embodiments, the samples are “mixture” samples, which comprise nucleic acids from more than one subject or individual. In some embodiments, the methods provided herein comprise purifying the sample or purifying the nucleic acid(s) from the sample. In some embodiments, the sample is purified nucleic acid.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, in some embodiments the term “substantial complementarity” means that a primer member of a primer pair comprises between about 70%-100%, or between about 80-100%, or between about 90-100%, or between about 95-100%, or between about 99-100% complementarity with the conserved binding sequence of a given nucleic acid or the nucleic acid from a given sample. These ranges of complementarity and identity are inclusive of all whole or partial numbers embraced within the recited range numbers. For example, and not limitation, 75.667%, 82%, 91.2435% and 97% complementarity or sequence identity are all numbers that fall within the above recited range of 70% to 100%, therefore forming a part of this description. A “system” in the context of analytical instrumentation refers a group of objects and/or devices that form a network for performing a desired objective.

### DETAILED DESCRIPTION

In some embodiments, the present invention provides compositions and methods for determining the methylation status of nucleic acids (e.g. RNA or DNA (e.g. GC rich promoter DNA)). In some embodiments the present invention provides isolating nucleic acid (e.g. genomic DNA) from a subject or sample and treating the nucleic acid (e.g. DNA) with a bisulfite solution to convert unmethylated CpG residues to UpG. Methylated CpG residues (e.g. methylated C) are not converted. In some embodiments, the nucleic acid (e.g. DNA) is amplified (e.g. PCR amplification) using primers designed to flank the region of interest. In some embodiments, amplification (e.g. PCR) results in unmethylated C's being converted to T's, while methylated C's remain C's. In some embodiments, mass spectrometry is utilized to determine the mass and/or base composition of the amplicon. In some embodiments, the mass and/or base composition is used to determine the methylation status (e.g., the location and/or degree of methylation) of the region of interest. In some embodiments, the differences in the base composition of the probe region of the amplicon relative to the canonical unmethylated sequence of the probe region is used to determine the extent of methylation.

Bisulfite ion (IUPAC: hydrogen sulfite) is the ion HSO₃⁻. Salts containing the HSO₃⁻ ion are known as bisulfites or as sulfite lyes (e.g. sodium bisulfite is NaHSO₃). In some embodiments, bisulfate used is added to reactions as a bisulfate salt (e.g. sodium bisulfate). In some embodiments, bisulfite treatment of nucleic acid (e.g. DNA) is used to determine its pattern of methylation or methylation status. In some embodiments, bisulfite treatment of nucleic acid (e.g. DNA) modifies the nucleic acid. In some embodiments, treatment of nucleic acid (e.g. DNA) with bisulfite converts cytosine residues to uracil, but leaves 5-methylcytosine residues unmodified. Thus, bisulfite treatment introduces specific changes in the DNA base composition that depend on the methylation status of individual cytosine residues. In some embodiments, bisulfate treatment yields single-nucleotide resolution information about the methylation status of a segment of DNA.

In some embodiments, nucleic acids comprise DNA and/or RNA. In some embodiments, nucleic acids are isolated and/or purified from a sample (e.g. a biological sample) or subject (e.g. human, model organism, etc.). In some embodiments, applicable nucleic acid isolation and purification techniques (e.g. cell lyses, ethanol precipitation, gel electrophoresis, column chromatography, phenol extraction, nuclease treatment, protease treatment, etc.) are known to those of skill in the art or are as described herein or within the references cited herein. In some embodiments, a biological sample includes, but is not limited to cells, cell lines, tissues, whole or partial organisms, clinical samples, blood samples, cell cultures, bacterial cells, viruses, animals (e.g. model organisms or other organisms of interest), mammals or humans, etc. Samples may be alive, non-replicating, dead, in a vegetative state, frozen, etc. In some embodiments, a subject comprises a human, non-human primate, mammal, rodent, bovine, porcine, equine, avian, feline, canine, non-mammal, etc. In some embodiments, nucleic acid comprises DNA.

In some embodiments, methods of the present invention comprise isolating nucleic acid (e.g. genomic DNA) from a subject (e.g. human) or sample (e.g. blood). In some embodiments, purified and/or isolated nucleic acid (e.g. DNA) is subjected to bisulfate treatment (e.g. reacting DNA with bisulfate). In some embodiments, following bisulfate treatment, the nucleic acid (e.g. DNA) is amplified (e.g. PCR). During new strand DNA synthesis, the presence of a U in the template strand results in an A being synthesized in the complementary position on the newly synthesized strand. The presence of a C^{m} in the template strand results in a G being synthesized in the complementary position on the newly synthesized strand. The presence of a G in the template strand results in a C being synthesized in the complementary position on the newly synthesized strand. The presence of an A in the template strand results in a T being synthesized in the complementary position on the newly synthesized strand. Therefore, in some embodiments, the presence of an unmethylated C, which is modified by bisulfate treatment to a U, will result in a T-A base pair in the amplified DNA. A methylated C, which is remains unmodified following bisulfate treatment, will result in a C-G pair in the amplified DNA. Therefore, bisulfate treatment, followed by amplification, results in different amplified nucleic acids depending upon the methylation status of cytosines in the nucleic acid. In some embodiments, the present invention provides compositions and methods for detecting the differences in the DNA that is the result of bisulfate treatment followed by amplification. In some embodiments, the present invention measures the base composition of the resulting DNA to determine the methylation status of the original nucleic acid.

Different nucleotides have different molecular masses (SEE Table 1.).

TABLE 1

| Nucleobase | Molecular Mass |
|---|---|
| A | 313.058 |
| T | 304.046 |
| C | 289.046 |
| G | 329.052 |

In some embodiments, the present invention provides compositions and methods for ascertaining the base composition of a nucleic acid molecule by determining the molecular weight the molecule. In some embodiments, the methyltation status of a nucleic acid molecule can be determined based on the base composition of the bisulfate treated and amplified nucleic acid molecule.

Particular embodiments of the mass-spectrum based detection methods are described in the following patents, patent applications and scientific publications, all of which are herein incorporated by reference as if fully set forth herein: U.S. Pat. Nos. 7,108,974; 7,217,510; 7,226,739; 7,255,992; 7,312,036; 7,339,051; US patent publication numbers 2003/0027135; 2003/0167133; 2003/0167134; 2003/0175695; 2003/0175696; 2003/0175697; 2003/0187588; 2003/0187593; 2003/0190605; 2003/0225529; 2003/0228571; 2004/0110169; 2004/0117129; 2004/0121309; 2004/0121310; 2004/0121311; 2004/0121312; 2004/0121313; 2004/0121314; 2004/0121315; 2004/0121329; 2004/0121335; 2004/0121340; 2004/0122598; 2004/0122857; 2004/0161770; 2004/0185438; 2004/0202997; 2004/0209260; 2004/0219517; 2004/0253583; 2004/0253619; 2005/0027459; 2005/0123952; 2005/0130196 2005/0142581; 2005/0164215; 2005/0266397; 2005/0270191; 2006/0014154; 2006/0121520; 2006/0205040; 2006/0240412; 2006/0259249; 2006/0275749; 2006/0275788; 2007/0087336; 2007/0087337; 2007/0087338 2007/0087339; 2007/0087340; 2007/0087341; 2007/0184434; 2007/0218467; 2007/0218467; 2007/0218489; 2007/0224614; 2007/0238116; 2007/0243544; 2007/0248969; 20080160512, 20080311558, 20090004643, 20090047665, 20090125245, 20090148829, 20090148836, 20090148837, 20090182511, WO2002/070664; WO2003/001976; WO2003/100035; WO2004/009849; WO2004/052175; WO2004/053076; WO2004/053141; WO2004/053164; WO2004/060278; WO2004/093644; WO 2004/101809; WO2004/111187; WO2005/023083; WO2005/023986; WO2005/024046; WO2005/033271; WO2005/036369; WO2005/086634; WO2005/089128; WO2005/091971; WO2005/092059; WO2005/094421; WO2005/098047; WO2005/116263; WO2005/117270; WO2006/019784; WO2006/034294; WO2006/071241; WO2006/094238; WO2006/116127; WO2006/135400; WO2007/014045; WO2007/047778; WO2007/086904; WO2007/100397; WO2007/118222, Ecker et al. (2005) “The Microbial Rosetta Stone Database: A compilation of global and emerging infectious microorganisms and bioterrorist threat agents” BMC Microbiology 5(1):19; Ecker et al. (2006) “The Ibis T5000 Universal Biosensor: An Automated Platform for Pathogen Identification and Strain Typing” JALA 6 (10:341-351; Ecker et al. (2006) “Identification of Acinetobacter species and genotyping of Acinetobacter baumannii by multilocus PCR and mass spectrometry” J Clin Microbiol. 44(8):2921-32; Ecker et al. (2005) “Rapid identification and strain-typing of respiratory pathogens for epidemic surveillance” Proc Natl Acad Sci USA. 102(22):8012-7; Hannis et al. (2008) “High-resolution genotyping of Campylobacter species by use of PCR and high-throughput mass spectrometry” J Clin Microbiol. 46(4):1220-5; Blyn et al. (2008) “Rapid detection and molecular serotyping of adenovirus by use of PCR followed by electrospray ionization mass spectrometry” J Clin Microbiol. 46(2):644-51; Sampath et al. (2007) “Global surveillance of emerging Influenza virus genotypes by mass spectrometry” PLoS ONE 2 (5):e489; Sampath et al. (2007) “Rapid identification of emerging infectious agents using PCR and electrospray ionization mass spectrometry” Ann N Y Acad. Sci. 1102:109-20; Hall et al. (2005) “Base composition analysis of human mitochondrial DNA using electrospray ionization mass spectrometry: a novel tool for the identification and differentiation of humans” Anal Biochem. 344(1):53-69; Hofstadler et al. (2003) “A highly efficient and automated method of purifying and desalting PCR products for analysis by electrospray ionization mass spectrometry” Anal Biochem. 316:50-57; Hofstadler et al. (2006) “Selective ion filtering by digital thresholding: A method to unwind complex ESI-mass spectra and eliminate signals from low molecular weight chemical noise” Anal Chem. 78(2):372-378; and Hofstadler et al. (2005) “TIGER: The Universal Biosensor” Int J Mass Spectrom. 242(1):23-41, each of which is herein incorporated by reference in its entirety.

In some embodiments, amplicons amenable to molecular mass determination are of a length, size or mass compatible with a particular mode of molecular mass determination, or compatible with a means of providing a fragmentation pattern in order to obtain fragments of a length compatible with a particular mode of molecular mass determination. Such means of providing a fragmentation pattern of an amplicon include, but are not limited to, cleavage with restriction enzymes or cleavage primers, sonication or other means of fragmentation. Thus, in some embodiments, bioagent identifying amplicons are larger than 200 nucleobases and are amenable to molecular mass determination following restriction digestion. Methods of using restriction enzymes and cleavage primers are well known to those with ordinary skill in the art.

In some embodiments, amplicons are obtained using the polymerase chain reaction (PCR). Other amplification methods may be used such as ligase chain reaction (LCR), low-stringency single primer PCR, and multiple strand displacement amplification (MDA). (Michael, S F., Biotechniques (1994), 16:411-412 and Dean et al., Proc Natl Acad Sci USA (2002), 99, 5261-5266).

Synthesis of primers is well known and routine in the art. The primers may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed.

In some embodiments, an amplicon is produced using only a single primer (either the forward or reverse primer of any given primer pair), provided an appropriate amplification method is chosen, such as, for example, low stringency single primer PCR (LSSP-PCR). In some embodiments, an amplicon is produced from a oligonucleotide primer pair.

In some embodiments, the oligonucleotide primers hybridize to conserved regions of nucleic acid. One with ordinary skill in the art of design of amplification primers will recognize that a given primer need not hybridize with 100% complementarity in order to effectively prime the synthesis of a complementary nucleic acid strand in an amplification reaction. The primers may comprise at least 70%, at least 75%, at least 80%, at least 85%, at least 90%, at least 95% or at least 99% sequence complementarity with the target sequence to be primed.

Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). In some embodiments, complementarity of primers with respect to the conserved priming regions sample nucleic acid, is between about 70% and about 80%. In other embodiments, homology, sequence identity or complementarity, is between about 80% and about 90%. In yet other embodiments, homology, sequence identity or complementarity, is at least 90%, at least 92%, at least 94%, at least 95%, at least 96%, at least 97%, at least 98%, at least 99% or is 100%.

In some embodiments, the oligonucleotide primers are 10 to 35 nucleobases in length (10 to 35 linked nucleotide residues). These embodiments comprise oligonucleotide primers 10, 11, 12, 13, 14, 15, 16, 17, 18, 19, 20, 21, 22, 23, 24, 25, 26, 27, 28, 29, 30, 31, 32, 33, 34 or 35 nucleobases in length, or any range therewithin. One of skill in the art understands that suitable primer lengths outside of this range may also be sued with the present invention.

In some embodiments, any given primer comprises a modification comprising the addition of a non-templated T residue to the 5′ end of the primer (i.e., the added T residue does not necessarily hybridize to the nucleic acid being amplified). The addition of a non-templated T residue has an effect of minimizing the addition of non-templated A residues as a result of the non-specific enzyme activity of, e.g., Taq DNA polymerase (Magnuson et al., Biotechniques, 1996: 21, 700-709), an occurrence which may lead to ambiguous results arising from molecular mass analysis.

In some embodiments, non-template primer tags are used to increase the melting temperature (Tₘ) of a primer-template duplex in order to improve amplification efficiency. A non-template tag is at least three consecutive A or T nucleotide residues on a primer which are not complementary to the template. In any given non-template tag, A can be replaced by C or G and T can also be replaced by C or G. Although Watson-Crick hybridization is not expected to occur for a non-template tag relative to the template, the extra hydrogen bond in a G-C pair relative to an A-T pair confers increased stability of the primer-template duplex and improves amplification efficiency for subsequent cycles of amplification when the primers hybridize to strands synthesized in previous cycles.

In other embodiments, propynylated tags may be used in a manner similar to that of the non-template tag, wherein two or more 5-propynylcytidine or 5-propynyluridine residues replace template matching residues on a primer. In other embodiments, a primer contains a modified internucleoside linkage such as a phosphorothioate linkage, for example.

In some embodiments, the primers contain mass-modifying tags. Reducing the total number of possible base compositions of a nucleic acid of specific molecular weight provides a means of avoiding a possible source of ambiguity in the determination of base composition of amplicons. Addition of mass-modifying tags to certain nucleobases of a given primer will result in simplification of de novo determination of base composition of a given amplicon from its molecular mass.

In some embodiments, the mass modified nucleobase comprises one or more of the following: for example, 7-deaza-2′-deoxyadenosine-5-triphosphate, 5-iodo-2′-deoxyuridine-5′-triphosphate, 5-bromo-2′-deoxyuridine-5′-triphosphate, 5-bromo-2′-deoxycytidine-5′-triphosphate, 5-iodo-2′-deoxycytidine-5′-triphosphate, 5-hydroxy-2′-deoxyuridine-5′-triphosphate, 4-thiothymidine-5′-triphosphate, 5-aza-2′-deoxyuridine-5′-triphosphate, 5-fluoro-2′-deoxyuridine-5′-triphosphate, O6-methyl-2′-deoxyguanosine-5′-triphosphate, N2-methyl-2′-deoxyguanosine-5′-triphosphate, 8-oxo-2′-deoxyguanosine-5′-triphosphate or thiothymidine-5′-triphosphate. In some embodiments, the mass-modified nucleobase comprises ¹⁵N or ¹³C or both ¹³N and ¹³C.

In some embodiments, the molecular mass an amplicon is determined by mass spectrometry. Mass spectrometry is intrinsically a parallel detection scheme without the need for radioactive or fluorescent labels, because an amplicon is identified by its molecular mass. The current state of the art in mass spectrometry is such that less than femtomole quantities of material can be analyzed to provide information about the molecular contents of the sample. An accurate assessment of the molecular mass of the material can be quickly obtained, irrespective of whether the molecular weight of the sample is several hundred, or in excess of one hundred thousand atomic mass units (amu) or Daltons.

In some embodiments, intact molecular ions are generated from amplicons using one of a variety of ionization techniques to convert the sample to the gas phase. These ionization methods include, but are not limited to, electrospray ionization (ESI), matrix-assisted laser desorption ionization (MALDI) and fast atom bombardment (FAB). Upon ionization, several peaks are observed from one sample due to the formation of ions with different charges. Averaging the multiple readings of molecular mass obtained from a single mass spectrum affords an estimate of molecular mass of the amplicon. Electrospray ionization mass spectrometry (ESI-MS) is particularly useful for very high molecular weight polymers such as proteins and nucleic acids having molecular weights greater than 10 kDa, since it yields a distribution of multiply-charged molecules of the sample without causing a significant amount of fragmentation.

The mass detectors used include, but are not limited to, Fourier transform ion cyclotron resonance mass spectrometry (FT-ICR-MS), time of flight (TOF), ion trap, quadrupole, magnetic sector, Q-TOF, and triple quadrupole.

In some embodiments, primers are designed to conserved sequences flanking a variable region (e.g., variable in the position or number of methylated bases), such that amplicons produced from the primers are able to differentiate two or more target nucleic acids based on differences in mass or base composition from the variable region.

### EXPERIMENTAL

### Example 1

### Exemplary Embodiment

The following example is provided in order to demonstrate and further illustrate certain preferred embodiments and aspects of the present invention and is not to be construed as limiting the scope thereof.

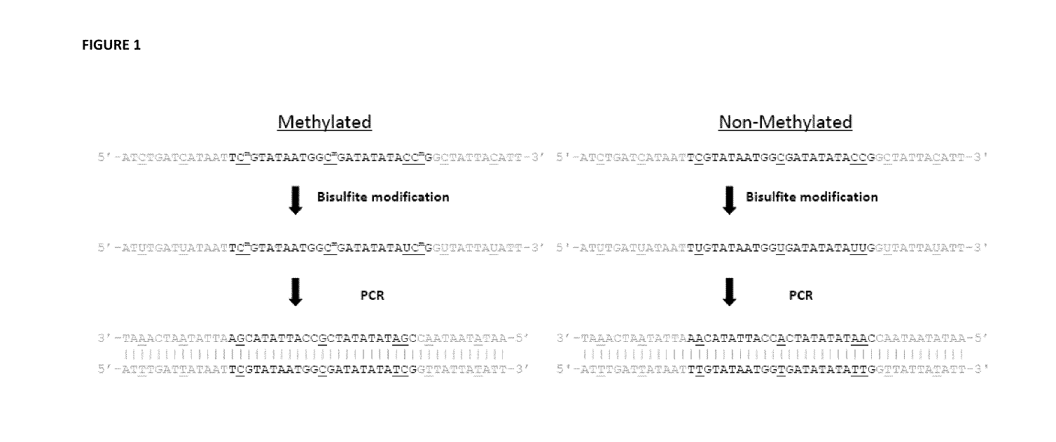

In an exemplary embodiment, the present invention provides compositions (e.g. primers, instruments, and reagents) and methods for detecting the methylation status of a DNA molecule. The following example demonstrates the differential effect of methods of the present invention on methylated cytosine and unmethylated cytosine (SEE FIG. 1). A methylated and unmethylated DNA molecule are isolated and purified, or provided in a substantially pure form. The methylated DNA contains three 5-methylcytosine residues and one unmethylated cytosine residue in the probe region, while the non-methylated DNA contains 4 unmethylated cytosines in the probe region. Each DNA sample is subjected to bisulfate modification according as described herein. Reaction of the bisulfate with the DNA results in conversion of unmethylated cytosines to uracil residues, while 5-methylcytosines do not react with bisulfate and remain 5-methycytosine residues (SEE FIG. 1). The DNA samples are then amplified by PCR using primer oligonucleotides which are complementary to primer binding regions which flank the regions containing the methylated/unmethylated bases. Amplification of the bisulfate-reacted DNA samples by PCR results in the synthesis of complementary double stranded DNA from the bisulfate-modified methylated and non-methylated DNA templates. PCR amplification results in guanine residues pairing with the template 5-methylcytosine. Cytosine pairs with the guanine residues in the amplified DNA, resulting in newly synthesized G-C pairs at the position of the unmodified 5-methylcytosines in the amplicons. PCR amplification results in adenine residues pairing with the template uracil residues (uracil is the result of bisulfate modification of unmethylated cytosine). Thymine pairs with the adenine residues in the amplified DNA, resulting in newly synthesized A-T pairs at the position of the bisulfate modified cytosines in the amplicon. Mass determination of the amplicons by mass spectrometry indicates an amplicon mass of 14201.344 g/mol for the methylated DNA sample and 14198.362 g/mol for the non-methylated DNA sample. These molecular masses are used to determine a base composition the double stranded amplicons of A₁₅-T₁₅-G₈-C₈ for the methylated DNA sample and of A₁₈-T₁₈-G₅-C₅ for the non-methylated DNA sample. The difference in base composition reveals the presence of three methylated cytosines in the methylated DNA sample, and no methylated cytosines in the non-methylated DNA sample.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, internet web sites, and the like) cited in the present application is incorporated herein by reference in its entirety.

We claim:

1. A method of determining the methylation status of a nucleic acid, the method comprising:
(a) reacting said nucleic acid with bisulfate;
(b) amplifying one or more segments said nucleic acid using at least one purified oligonucleotide primer pair to produce an amplification product; and
(c) determining the mass and/or base composition of said amplification product, thereby determining said methylation status of said nucleic acid.

2. The method of claim 1, wherein said nucleic acid comprises DNA.

3. The method of claim 1, wherein said nucleic acid is GC-rich.

4. The method of claim 1, wherein said nucleic acid comprises a DNA promoter.

5. The method of claim 1, wherein bisulfate reacts with unmethylated cytosine residues, converting them to uracil residues.

6. The method of claim 1, wherein bisulfate does not react with methylated cytosine residues, leaving them as 5-methylcytosine.

7. The method of claim 1, wherein amplifying one or more segments said nucleic acid comprises PCR.

8. The method of claim 1, wherein (c) comprises detecting a molecular mass of said amplification product.

9. The method of claim 1, wherein (c) comprises determining a base composition of said amplification product, wherein said base composition identifies the number of A residues, C residues, T residues, G residues, U residues, analogs thereof and/or mass tag residues thereof in said amplification product, whereby said base composition indicates the methylation status of said nucleic acid.

10. The method of claim 9, comprising comparing said base composition of said amplification product to calculated or measured base compositions of amplification products present in a database with the proviso that sequencing of said amplification product is not used to indicate the methylation status, wherein a match between the determined base composition and the calculated or measured base composition in said database indicates methylation status.

11. The method of claim 9, comprising comparing said base composition of said amplification product to the base composition of a control nucleic acid with the proviso that sequencing of said amplification product is not used to indicate the methylation status, wherein differences in mass between the determined base composition and control base composition indicates methylation status.

12. The method of claim 1, further comprising an initial step of isolating said nucleic acid from a subject or sample.

13. A system for determining the methylation status of a nucleic acid, the system comprising:
(a) instrumentation for calculating a molecular mass of a nucleic acid molecule; and
(b) a database comprising masses or base compositions of known bisulfate converted nucleic acid molecules.

14. The system of claim 13, further comprising (c) reagents for bisulfate treatment of a nucleic acid molecule.


SEQUENCE LISTING (6 sequences)

SEQ ID NO: 1 - DNA, 47 bp, Artificial Sequence
  Description of Artificial Sequence Synthetic oligonucleotide
atctgatcataattcgtataatggcgatatataccggctattacatt

SEQ ID NO: 2 - DNA, 47 bp, Artificial Sequence; identical to SEQ ID NO: 1
  Description of Artificial Sequence Synthetic oligonucleotide

SEQ ID NO: 3 - DNA, 47 bp, Artificial Sequence
  Description of Artificial Sequence Synthetic oligonucleotide
atutgatuataattcgtataatggcgatatataucggutattauatt

SEQ ID NO: 4 - DNA, 47 bp, Artificial Sequence
  Description of Artificial Sequence Synthetic oligonucleotide
atutgatuataattugtataatggugatatatauuggutattauatt

SEQ ID NO: 5 - DNA, 47 bp, Artificial Sequence
  Description of Artificial Sequence Synthetic oligonucleotide
atttgattataattcgtataatggcgatatatatcggttattatatt

SEQ ID NO: 6 - DNA, 47 bp, Artificial Sequence
  Description of Artificial Sequence Synthetic oligonucleotide
atttgattataatttgtataatggtgatatatattggttattatatt